SEQUENCE LISTING (35 sequences)

SEQ ID NO: 1 - DNA, 20 bp, Artificial Sequence
  guideRNA sequence
aggattgatgttcaggcttc

SEQ ID NO: 2 - DNA, 25 bp, Artificial Sequence
  Primer sequence
gagctatctgtctttaggcctggag

SEQ ID NO: 3 - DNA, 24 bp, Artificial Sequence
  Primer sequence
caacctggcctacatagtgagctc

SEQ ID NO: 4 - DNA, 25 bp, Artificial Sequence
  Primer sequence
gagctatctgtctttaggcctggag

SEQ ID NO: 5 - DNA, 20 bp, Artificial Sequence
  Primer sequence
aggattgatgttcaggcttc

SEQ ID NO: 6 - DNA, 133 bp, Artificial Sequence
  Template sequence
tgcatagcccttactggcctcccccatgttattctctgaagccggaagccgtgagcggctggcggctgttcaagaagattagctgaacatcaatcctttgatggaacctcaaagtcctatagtcctaagtgac

SEQ ID NO: 7 - DNA, 20 bp, Artificial Sequence
  Primer sequence
gtgagcggctggcggctgtt

SEQ ID NO: 8 - DNA, 24 bp, Artificial Sequence
  Primer sequence
caacctggcctacatagtgagctc

SEQ ID NO: 9 - DNA, 24 bp, Artificial Sequence
  Primer sequence
caacctggcctacatagtgagctc

SEQ ID NO: 10 - DNA, 20 bp, Artificial Sequence
  guideRNA target sequence
ctgcgctgtctagagtccct

SEQ ID NO: 11 - DNA, 20 bp, Artificial Sequence
  guideRNA target sequence
caaaagtatggccgcttctg

SEQ ID NO: 12 - DNA, 101 bp, Artificial Sequence
  Template oligo sequence
gaccaagaggaaaaagcagactttgaaaattgggcatggagggactctagtgagcgcagcccgaggagttctggttgttggaattggaagcggaacaaaaa

SEQ ID NO: 13 - DNA, 200 bp, Artificial Sequence
  Template oligo sequence
cagcgtgcacctccacgatgaaacaggtctgggctacaaaagtatggccgcttctgtgagcggctggcggctgttcaagaagattagcgactacaaagaccatgacggtgattataaagatcatgacatcgattacaaggatgacgatgacaaggccgcttctgaggcggcggtggtgtcttcgccgtctttgaaaacag

SEQ ID NO: 14 - DNA, 20 bp, Artificial Sequence
  Primer sequence
gttttgaaaatgccatcccc

SEQ ID NO: 15 - DNA, 20 bp, Artificial Sequence
  Primer sequence
agaaatagctacttttatgt

SEQ ID NO: 16 - DNA, 20 bp, Artificial Sequence
  guideRNA target sequence
tccccttttcctcccaagtt

SEQ ID NO: 17 - DNA, 20 bp, Artificial Sequence
  guideRNA target sequence
tttctctcatagaagctgga

SEQ ID NO: 18 - DNA, 136 bp, Artificial Sequence
  Template oligo sequence
ctagtaatgaggtcatagtctcttaacatgtaaagtttgtataatactttgtgagcggctggcggctgttcaagaagattagcttaatagaagctggaatgccttctccagaatggaccaagaggaaaaagcagac

SEQ ID NO: 19 - DNA, 20 bp, Artificial Sequence
  guideRNA target sequence
gttgtggtgatctccaacat

SEQ ID NO: 20 - DNA, 102 bp, Artificial Sequence
  Template oligo sequence
cccagctcagtccccactccctccgcagacccactccttgccagttgtgtagatctccaacatctgtcagatgccaaatgcctgggcgtccatcctgtggta

SEQ ID NO: 21 - DNA, 20 bp, Artificial Sequence
  Primer sequence
gcagcaggtgtggtttatgg

SEQ ID NO: 22 - DNA, 20 bp, Artificial Sequence
  Primer sequence
accatccctcatctaaacaa

SEQ ID NO: 23 - DNA, 20 bp, Artificial Sequence
  guideRNA target sequence
tgcgctacctcccccatgtg

SEQ ID NO: 24 - DNA, 100 bp, Artificial Sequence
  Template oligo sequence
cccagctcagtccccactccctccgcagacccactccttgccagttgtggtgatctccaacatctgtcagatgccaaatgcctgggcgtccatcctgtgg

SEQ ID NO: 25 - DNA, 133 bp, Artificial Sequence
  Template oligo sequence
ccctcacctttgacatggagttgacctcggagtgcgctacctcccccatggtgagcggctggcggctgttcaagaagattagctgaggagctgagaacggaagctgcagaaagatacgactgaggcgcctacc

SEQ ID NO: 26 - RNA, 20 nt, Artificial Seqeunce
  guideRNA sequence
uuguugaguaggagaaucuc

SEQ ID NO: 27 - RNA, 20 nt, Artificial Sequence
  guideRNA Sequence
uuuaaaaaaugaguguggca

SEQ ID NO: 28 - RNA, 20 nt, Artificial Sequence
  guideRNA Sequence
ucaaggccacccugggcaac

SEQ ID NO: 29 - RNA, 20 nt, Artificial Sequence
  guideRNA Sequence
gcuuagcguauuuuauguug

SEQ ID NO: 30 - RNA, 20 nt, Artificial Sequence
  guideRNA Sequence
acaccacagacuuuacaucc

SEQ ID NO: 31 - DNA, 100 bp, Artificial Sequence
  Template oligo Sequence
gatcatggattcaagaaataaatgtaacggaaaagaattggtatccacaacataaaatacgctatgctaagagaaagttagcaggggccaacccagctgt

SEQ ID NO: 32 - DNA, 151 bp, Artificial Sequence
  Template oligo Sequence
cagaccccaatatccttggcaggacttgggaaggatgggagccctggatgcgggaccacatggtgctgcacgagtacgtgaacgccgccggcatcacataataaagtctgtggtgtcaccaatcataaagcattctgtctccgagaggacc

SEQ ID NO: 33 - DNA, 29 bp, Artificial Sequence
  Primer Sequence
caaactttgaattcacagtcatgagtgac

SEQ ID NO: 34 - DNA, 23 bp, Artificial Sequence
  Primer sequence
gaggtcctctcggagacagaatg

SEQ ID NO: 35 - DNA, 642 bp, Artificial Sequence
  GFP1-10 sequence
tccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaataa

(19) United States
(12) Patent Application Publication
(10) Pub. No.: US 2022/0177922 A1
(43) Pub. Date: Jun. 9, 2022

(54) HIGH-THROUGHPUT GENE-EDITING TECHNIQUE

(71) Applicants: National University Corporation Tokyo Medical and Dental University, Tokyo (JP); SYSMEX CORPORATION, Kobe-shi, Hyogo (JP)

(72) Inventors: Hiroshi ASAHARA, Tokyo (JP); Ryota KURIMOTO, Tokyo (JP); Takahide MATSUSHIMA, Tokyo (JP); Hiroki TSUTSUMI, Tokyo (JP)

(73) Assignees: National University Corporation Tokyo Medical and Dental University, Tokyo (JP); SYSMEX CORPORATION, Kobe-shi, Hyogo (JP)

(21) Appl. No.: 17/436,208

(22) PCT Filed: Mar. 9, 2020
(86) PCT No.: PCT/JP2020/009987

§ 371 Date: Oct. 27, 2021

(30) Foreign Application Priority Data
Mar. 7, 2019 (JP) 2019-041177

(51) Int. Cl. C12N 15/90

(52) U.S. Cl. CPC C12N 15/90

(57) ABSTRACT

One of the purposes of the present invention is to provide a method for producing a gene-edited tissue stem cell in an undifferentiated state within a short period of time. Another one of the purposes of the present invention is to provide a method for treating and/or preventing a disease using a gene-edited tissue stem cell in an undifferentiated state. A gene-edited tissue stem cell is produced by a method comprising a step for editing the gene sequence of a target gene in tissue stem cells, and a step for selecting the tissue stem cells having an edited gene sequence, and optionally, further comprising a step for adding a tag to the gene sequence of the target gene and/or a step for activating gene expression of the target gene in the tissue stem cells. The tissue stem cell thus produced is used in treating/preventing diseases.

### TECHNICAL FIELD

The present invention relates to a method of gene editing, and specifically to a method of short-term gene editing of tissue stem cells.

### BACKGROUND ART

Genome editing is a technology that uses site-specific nucleases to modify target genes. For the nucleases, ZFN, TALEN, CRISPR/Cas9 and such are used. Compared to conventional genetic engineering and gene therapy, this technology has a much wider range of applications, and the development of therapies based on genome editing technology is underway (Non-patent Document 1).

For example, CRISPR Therapeutics (Switzerland) is developing a treatment (CTX001) for sickle cell anemia using genome editing (Non-patent Documents 2 and 3). Sickle cell anemia is caused by mutations in the HBB gene, which provides instructions for making hemoglobin, a molecule in red blood cells that carries oxygen. In sickle cell anemia, this mutation results in a deficiency of hemoglobin, reducing the oxygen-carrying function of red blood cells.

CTX001 aims to use gene editing technology to alter a gene and increase the production of fetal hemoglobin (HbF) in the patient's red blood cells. Fetal hemoglobin is a type of hemoglobin that exists naturally in newborns and is later replaced by the adult form of hemoglobin. Occasionally, however, fetal hemoglobin survives into adulthood, protecting people from sickle cell anemia and β-thalassemia.

For the treatment, hematopoietic stem cells, which are bone marrow-derived cells that give rise to the red blood cells and white blood cells that make up the blood, are harvested from the patient and then genetically modified to produce high levels of fetal hemoglobin. More specifically, the BCL11A gene, a transcriptional repressor of HbF, is disrupted by genetic modification. The processed cells are then returned to the patient's body, where it is believed that the patient's body can produce large numbers of red blood cells containing fetal hemoglobin, thereby overcoming the hemoglobin deficiency caused by the disease.

However, in the treatment of diseases of hematology, immunology, cancer, and such, many problems remain, such as the efficiency of gene editing on tissue stem cells and the difficulty of cell selection, and there are still many inadequacies in the medical application of gene editing technology.

### CITATION LIST

### Non-Patent Documents

Non-patent Document 1: Dunbar et al., Gene therapy comes of age. Science. 2018; 359:175-184. DOI: 10.1126/science.aan4672
Non-patent Document 2: Antony et al., CRISPR/Cas9 system: a promising technology for the treatment of inherited and neoplastic hematological diseases, Adv Cell Gene Ther. 2018; 1:e10. DOI: 10.1002/acg2.10
Non-patent Document 3: Lin M I, et al. CRISPR/Cas9 Genome Editing to Treat Sickle Cell Disease and B-Thalassemia: Re-Creating Genetic Variants to Upregulate Fetal Hemoglobin Appear Well-Tolerated, Effective and Durable. Blood. 2017; 130:284.

### SUMMARY OF THE INVENTION

### Problems to be Solved by the Invention

One of the objectives of the present invention is to provide a method for producing gene-edited tissue stem cells in an undifferentiated state within a short period of time. Another objective of the present invention is to provide a method for treating and/or preventing a disease using gene-edited undifferentiated tissue stem cells.

### Means for Solving the Problems

The present inventors have developed a method for producing gene-edited tissue stem cells in an undifferentiated state within a short period of time by applying genome editing technology. By using this method, it is possible to provide a method for treating and/or preventing diseases using gene-edited undifferentiated tissue stem cells. More specifically, the present invention encompasses the embodiments below.

### Embodiment 1

A method for producing a gene-edited tissue stem cell, comprising editing the gene sequence of a target gene in the tissue stem cell and selecting the tissue stem cell with the edited gene sequence.

### Embodiment 2

The method according to embodiment 1, further comprising adding a tag to the gene sequence of the target gene and/or activating gene expression of the target gene in the tissue stem cell.

### Embodiment 3

The method according to embodiment 1 or 2, wherein the tissue stem cell is a tissue stem cell isolated from a patient.

### Embodiment 4

The method according to any one of embodiments 1-3, wherein the editing of the gene sequence is performed ex vivo.

### Embodiment 5

The method according to any one of embodiments 1-4, wherein the editing of the gene sequence is performed using a CRISPR/Cas system or a TALEN system.

### Embodiment 6

The method according to any one of embodiments 1-5, wherein the target gene is a gene that is not steadily expressed in tissue stem cells.

### Embodiment 7

The method according to any one of embodiments 2-6, wherein the activation of the gene expression is performed using the CRISPRa system or the TALEN effector system.

### Embodiment 8

The method according to any one of embodiments 2-7, wherein the tissue stem cell with the edited gene sequence is selected using the tag added to the gene sequence.

### Embodiment 9

The method according to any one of embodiments 2-8, wherein the step of editing the gene sequence of the target gene in the tissue stem cell and the step of adding a tag to the gene sequence of the target gene are performed simultaneously.

### Embodiment 10

The method according to any one of embodiments 1-9, wherein the tissue stem cell with the edited gene sequence is selected within 24 hours of isolating the cell from the patient.

### Embodiment 11

The method according to any one of embodiments 1-10, wherein the selected tissue stem cell maintains an undifferentiated state.

### Embodiment 12

The method according to any one of embodiments 1-11, further comprising proliferating the selected tissue stem cell.

### Embodiment 13

The method according to any one of embodiments 1-12, further comprising transplanting the selected tissue stem cell into the patient.

### Embodiment 14

The method according to any one of embodiments 1-13, wherein the tissue stem cell with the edited gene sequence is a cell for use in treating a disease.

### Embodiment 15

The method according to embodiment 14, wherein the disease is a hematological disease or an immunological disease.

### Embodiment 16

The method according to embodiment 15, wherein the hematological disease or immunological disease is selected from the group consisting of ADA deficiency, X-linked severe combined immunodeficiency (SCID), other SCID, Wiskott-Aldrich syndrome, chronic granulomatosis, leukocyte adhesion deficiency, familial hemophagocytic syndrome, X-linked hyper IgM syndrome, X-linked lymphoproliferative disease, X-linked agammaglobulinemia, hyper IgE syndrome, sickle cell disease, and ß-thalassemia.

### Embodiment 17

The method according to any one of embodiments 1-16, wherein the target gene is selected from the group consisting of ADA, IL2RG, WAS, CYBB, INTGB2, UNC13D, CD40L, SAP/SH2D1A, BTK, STAT3, and hemoglobin.

### Embodiment 18

A therapeutic agent for the treatment of a hematological disease or an immunological disease, comprising hematopoietic stem cells within 48 hours of isolation from an organism in which the gene sequence of the target gene has been edited.

### Embodiment 19

The therapeutic agent according to embodiment 18, wherein the hematological disease or immunological disease is selected from the group consisting of ADA deficiency, X-linked severe combined immunodeficiency (SCID), other SCID, Wiskott-Aldrich syndrome, chronic granulomatosis, leukocyte adhesion deficiency, familial hemophagocytic syndrome, X-linked hyper IgM syndrome, X-linked lymphoproliferative disease, X-linked agammaglobulinemia, hyper IgE syndrome, sickle cell disease, and ß-thalassemia.

### Embodiment 20

The therapeutic agent according to embodiment 18 or 19, wherein the target gene is selected from the group consisting of ADA, IL2RG, WAS, CYBB, INTGB2, UNC13D, CD40L, SAP/SH2D1A, BTK, STAT3, and hemoglobin.

### Embodiment 21

A method for producing a gene-edited tissue stem cell, comprising

editing the gene sequence of the target gene in the tissue stem cell,

adding a tag to the gene sequence of the target gene,

activating gene expression of the target gene in the tissue stem cell, and

selecting the tissue stem cell with the edited gene sequence,

wherein the step of editing the gene sequence of the target gene in the tissue stem cell and the step of adding a tag to the gene sequence of the target gene are performed simultaneously using a CRISPR/Cas system or a TALEN system.

### Embodiment 22

The method according to embodiment 21, wherein the target gene is a gene that is not steadily expressed in tissue stem cells, and

wherein the tag-labeled target protein of interest is produced by activating the gene expression of the target gene only in cells in which the gene sequence of the target gene has been edited and the tag has been added, and the tag is used to select the tissue stem cell with the edited gene sequence.

### Embodiment 23

The method according to embodiment 21 or 22, wherein the tissue cell is a tissue stem cell isolated from a patient, and the tissue stem cell with the edited gene sequence is selected within 48 hours of isolating the cell from the patient.

### Effects of the Invention

According to the present invention, gene-edited tissue stem cells in an undifferentiated state can be produced within a short period of time. Such tissue stem cells can then be used to treat and/or prevent diseases that have been difficult to intervene in the past.

### BRIEF DESCRIPTION OF THE DRAWINGS

FIG. 1 is an electrophoresis diagram (1% agarose gel) of the PCR products generated in Example 1 that have been treated with T7 endonuclease I (T7EI). From left to right, they are marker lane, DNA treated with T7EI, and DNA not treated with T7EI.

FIG. 2 shows the sequencing results of the cleaved PCR products generated in Example 1 (FASMAC, Big Dye terminator v3.1, 3130xl Genetic Analyzer). Upper row: sequencing results of uncut DNA. Lower row: sequencing results of truncated DNA.

FIG. 3 is an electrophoresis diagram (1% agarose gel) of the PCR products generated in Example 2. From left to right, they are marker lane, PCR products derived from unedited genome, PCR products derived from genome after genome editing, PCR products derived from genome after genome editing.

FIG. 4 shows the sequencing results of the PCR products generated in Example 2. It is clear that a tag sequence was introduced into the target site.

FIG. 5 shows the results of Western blotting with the Flag antibody of proteins extracted from Nano-Glo positive cells that have the introduced mutation in Example 4. It can be seen that the tag sequence has been introduced and functions as a peptide tag.

FIG. 6 shows the sequencing results of the PCR products generated in Example 4. It can be seen that the mutation was introduced into the target sequence at the target site.

FIG. 7 shows the results of lytic detection in Example 5, indicating that the luciferase activity was increased in the knock-in cells.

FIG. 8 shows the sequencing results of the cloned cells with the introduced mutation in Example 6. It can be seen that the mutation has been introduced into the target sequence.

FIG. 9 shows the results of lytic detection in Example 6. The Luciferase activity of HiBIT is increased by activation, indicating that the mutation has been introduced and the change is coordinated by gene activation.

FIG. 10 shows the sequencing results of the PCR products after reverting the mutation in Example 6. There is an overlap of the waveform in the target sequence, indicating that the cells with the mutation reverted are included.

FIG. 11 shows the results of agarose gel electrophoresis of the PCR products generated in Example 7. It can be seen that a tag sequence has been introduced into the target site.

FIG. 12 shows the sequencing results of the PCR products generated in Example 7. The results in FIGS. 11 and 12 show that there is an overlap of the tag sequence at the target site, indicating that the tag sequence was introduced with an efficiency of about 50%.

FIG. 13 is a fluorescence micrograph of cells in which GFP1-10 was introduced using AAV after introduction of the mutation in Example 7. It can be seen that GFP-11 was introduced into the cells and there are cells that have obtained the GFP fluorescence by binding with AAV-derived GFP1-10.

### MODE FOR CARRYING OUT THE INVENTION

Some embodiments of the present invention relate to a method of producing a gene-edited tissue stem cell that enables accurate selection of gene-edited tissue stem cells at single cell level in a short period of time. The method of the present invention comprises editing the gene sequence of a target gene in a tissue stem cell, selecting the gene-edited tissue stem cell, and possibly adding a tag to the gene sequence of the target gene and/or activating gene expression of the target gene in the tissue stem cell. Each of the steps is described in detail below.

Process of Editing the Gene Sequence of a Target Gene in a Tissue Stem Cell

Genome editing is a technology that introduces mutations (substitutions, insertions, and deletions) into an arbitrary genomic sequence using artificial restriction enzymes that can specifically cleave the target site of the genome. When a restriction enzyme causes a double-strand break at the target site, base pair deletions and insertions occur due to errors at the time of end joining during genome repair by NHEJ (Non-Homologous End Joining), a cellular repair mechanism, in the absence of a template DNA. As a result, the target gene is finally knocked out by frameshifting and such. On the other hand, in the presence of a template DNA, substitution or insertion of the template sequence into the target site occurs by HDR (Homology Dependent Repair), which is a repair mechanism (knock-in).

In the method of the present invention, the editing of the gene sequence can be either Knock-out, in which the gene is destroyed, or Knock-in, in which the sequence to be introduced or modified is replaced or inserted, but preferably it is a knock-in using the desired template sequence. As a result, for example, a sequence mutation that causes a disease is corrected to a normal sequence or a harmless sequence.

The main editing tools (correction tools) that can be used for editing the gene sequence of a target gene in the method of the present invention include, for example, a ZFN system (Zinc Finger Nucleases), a TALEN system (Transcription Activator Like Effector Nucleases), and a CRISPR/Cas system (Clustered Regularly Interspaced Short Palindromic Repeats/CRISPR Associated Protein 9). These systems are highly specific genome editing tools that consist of a nuclease responsible for breaking the DNA duplex of the genome, a guide molecule that directs the nuclease to the target site on the genome, and in some cases, a template DNA. The template DNA may be single-stranded DNA or double-stranded DNA.

The ZFN system (see, for example, Kim, Y. G., et al., 1996, Proc. Natl. Acad. Sci. USA 93:1156-1160) is an artificial chimeric protein composed of two functional domains, the Zinc Finger, which is one of the DNA-binding motifs present in the DNA-binding domain of many transcription factors, and a Fokl nuclease domain; and genome editing is carried out using a pair of ZFNs designed to sandwich the target sequence. In the ZFN system, the Zinc Finger, which is the DNA-binding domain, recognizes a specific gene sequence on the target gene; and the Fokl nuclease, which is the DNA-cleavage domain, induces double-strand breaks with high specificity.

The TALEN system (see, for example, Cermak T, et al., 2011, Nucleic Acids Res 39: e82), like ZFN, is an artificial chimeric protein which is a fusion of a Fokl nuclease domain to a DNA-binding domain (TAL effector (TALE)) that recognizes an arbitrary base sequence. The DNA-binding domain of TALEN recognizes 15 to 20 bases, and performs double-strand breaks using a pair of TALENs designed for the sense strand and antisense strand so as to sandwich a 14-20 base spacer that is the target sequence of FolI. The TALEN system enables more specific target sequence cleavage than ZFN, and is theoretically able to target arbitrary genomic sequence. The TALEN system is said to be superior to the CRISPR/CAS system in terms of suppressing off-target mutations.

The CRISPR/CAS system which is mainly represented by CRISPR/CAS9 (see, for example, Cong, L. et al., 2013, Science, 339, 819-823.) uses complementary binding of RNA and DNA for target sequence recognition, whereas the aforementioned ZFNs and TALENs use protein-DNA interactions for target sequence recognition. In the type II CRISPR/CAS system, in short, the guide RNA (a complex of crRNA and tracrRNA or a single-stranded sgRNA thereof) recruits the Cas9 nuclease, which is responsible for double-strand breaks, to the target sequence, and the CAS9-RNA complex excises a DNA sequence complementary to the guide RNA. In the type V CRISPR/Cas system, the RNA-dependent DNA nuclease Cpfl is used. The guide RNA in the type V CRISPR/Cas system consists only of crRNA. The CRISPR/Cas system is convenient in that multiple mutations can be introduced by introducing multiple guide RNAs because the guide RNA and the nuclease exist and function separately. For example, the components used in the CRISPR/Cas system (Cas9 gRNA, Cas9 protein, Cas9 plasmid, Cas9 carrier DNA, buffer, and such) and the components used in the CRISPR/Cas12a system (Cas12a (Cpfl) gRNA, Cas12a (Cpfl) protein, Cas12a (Cpfl) carrier DNA, buffer, and such) can be purchased from Integrated DNA Technologies (IDT), Inc. For the sgRNA design tool, for example, CRISPRdirect (http://crispr.dbcls.jp) can be used. For RNA synthesis and purification, for example, the mMessage mMachine (trademark) T7 UTRA Transcription Kit (Ambion, AM1345), MEGAclear (trademark) Transcription Clean-Up Kit (Ambion, AM1908), and such can be used.

For example, in the creation of guide RNA using Alt-RTM (IDT), Alt-RTM crRNA (100 μM, IDT) containing the target sequence, tracrRNA (100 μM, IDT, 1072532), Nuclease-free Duplex Buffer (IDT, 11-01-03-01) can be mixed at 3.58 μl, 3.58 μl, and 7.84 μl, respectively, placed at 95° C. for 5 minutes, and cooled at room temperature before use. In the preparation of sgRNA using a gRNA vector (Addgene No. 41824), the target sequence is introduced by the inverse PCR method to create a gRNA template DNA. RNA is synthesized with the mMessage mMachine T7 UTRA Transcription Kit (Ambion, AM1345), and purified to 100 μM using the MEGAclear Transcription Clean-Up Kit (Ambion, AM1908). When using a nuclease as a recombinant protein, SpCas9 (IDT, 1074182) and cpf1 (IDT, 1081069) can be diluted to 10 mg/ml and used. When using a nuclease as RNA, the RNA can be synthesized by the mMessage mMachine (trademark) T7 UTRA Transcription Kit (Ambion, AM1345) using SpCas9, SpCas-9-NG, dCas9, M-NMn-VP64 (Addgene #80425, #41816, #48676) on the vector as a template DNA, and purified by the ethanol precipitation method, for example, as described in the literature (Nishimasu H, et al. Science. 2018; 361:1259-1262. Esvelt K M, et al. Nat Methods. 2013; 10(11): 1116-1121.).

The ZFN system, the TALEN system, and the CRISPR/Cas system have been briefly described above, and improved versions of these systems and any other genome editing tools can also be used in the present invention. From the standpoint of specificity, it is preferable that gene sequence editing is performed using the CRISPR/Cas system or the TALEN system.

When using these genome editing tools, a plasmid expressing the guide molecule-nuclease complex or guide RNA and/or nuclease, or a virus packaged with the expression plasmid may be introduced into the target cells to express the guide molecule-nuclease complex or guide RNA and/or nuclease. Alternatively, one may introduce into the target cells the nuclease or guide molecule-nuclease complex as mRNA or protein, and the guide RNA as RNA, separately. In the case of introduction of an expression plasmid, the target cells may be treated with an agent that induces the expression. In terms of off-target risk due to continued expression of the nuclease by plasmid introduction, time lag for transcription and translation, and efficiency of introduction of the mutation, it is preferable to introduce the nuclease or guide molecule-nuclease complex as mRNA or protein and the guide RNA as RNA. Methods of introducing these RNAs, proteins, plasmids, or packaged viruses are not particularly limited and methods known in the art can be used; and non-limiting examples include calcium phosphate precipitation, lipofection, polymer capsules, microparticle guns, microinjection, and electroporation, and such. Electroporation is commonly used based on the balance between simplicity and introduction efficiency.

Electroporation to introduce RNA, protein or DNA into cells can be performed, for example, using a Nepa 21 pulse generator (Nepa Gene). The power transmission is, for example, two rectangular electrical pulses (225 V, 2 ms wide, 50 ms apart), followed by five pulses (30 V, 50 ms wide, 50 ms apart). The electroporated mixture can be washed by adding 2 ml of HBSS+ medium and centrifuging at 800 rpm for 3 minutes. The cells may be cultured for about 24 hours in the same medium used for collection of hematopoietic stem cells. Furthermore, by detecting GFP signals or other signals at the single cell level, it is possible to select cells in which gene editing has been performed accurately.

When handling tissue stem cells, it is desirable to have a work space suitable for RNA and protein under sterilization and an SPF section as an animal testing facility. In addition, equipment for detecting and selecting luminescent tags may be suitably used as a means for efficiently extracting cells into which mutations have been introduced.

Process of Selecting Out a Tissue Stem Cell with the Edited Gene Sequence

The method of the present invention comprises a step of selecting, at the single cell level, cells in which the target gene has been edited from a pool of tissue stem cells after editing of the target gene in the target cell. The method for selecting cells in which the target gene has been edited includes a method based on the nucleic acid sequence of the edited target gene and a method based on the expression of the edited target gene. From the viewpoint of accurately selecting cells capable of normally expressing the edited target gene at the single cell level, it is preferable to use a method based on the expression of the target gene, such as by detecting the expression of a transiently activated target gene. For example, a system using fluorescence or phosphorescence detection can be used to detect the expression of the activated target gene.

Process of Activating the Gene Expression of a Target Gene in the Tissue Stem Cell

In many cases, the target gene is a gene that is expressed when tissue stem cells differentiate into corresponding tissue cells and perform various functions. In other words, in many cases, the target gene is a gene that is not steadily expressed in tissue stem cells. Therefore, in order to select a cell with the edited target gene based on the expression of the target gene, the expression of the target gene must be transiently activated. Accordingly, in one embodiment, the method of the present invention may further comprise a step of activating gene expression of the target gene in a tissue stem cell. Such activation of gene expression can be transient. By this step, one can select at a single cell level only those cells that have been edited so that the target gene can be expressed normally. This step can also exclude cells that have undergone unintended editing, such as off-target mutations, in the process of editing the gene sequence described above.

Examples of the activation tool for transiently activating the target gene include a method in which a transcriptional activator (VP64, etc.) is allowed to act on a promoter region existing upstream of the target gene. In one embodiment, a method that can be performed substantially simultaneously with the process of editing the target gene described above or tagging the target gene described below is preferred.

Such activation tools include the CRISPRa (CRISPR activation) system and the TALEN effector system.

The CRISPRa system (see, for example, Silvana Konermann et al. Nature. 2015 Jan. 29; 6(7530583-588) is, in short, a system that recruits the dCas9-transcriptional activator complex at the transcription start site of the target gene to activate the expression of the target gene by introducing the dCas9-transcriptional activator complex, which is a fusion of a deadCas9 (dCas9) nuclease lacking DNA cleavage activity and a transcriptional activator (VP16, VP64, etc.), and a guide RNA targeting the promoter site of the target gene. As the CRISPRa system, expression vectors of the dCas9-transcription activator complex and the guide RNA, or viral particles packaged with the expression vectors can be introduced into the target cell. Alternatively, the dCas9-transcription activating factor complex may be introduced into the target cell as a protein and the guide RNA may be introduced into the target cell as RNA. In the present embodiment, from the viewpoint of rapid activation and safety, it is more preferable to separately introduce the dCas9-transcriptional activator complex as a protein and guide RNA as RNA into the target cell.

It is also desirable to use a TAL effector system in which a TAL DNA-binding domain targeting the promoter region of the target gene is fused to a transcriptional activator (VP16, VP64, etc.). As a TAL effector system design tool, for example, http://tale-nt.cac.cornell.edu/node/add/single-tale can be used. For example, with reference to the method described in the literature (Matsubara, Y, et al. Sci Rep, 4: 5043, 2014), the TAL effector is created into a mammalian expression vector (pcDNA-TAL-VP-64 (Addgene, 47107)) by the Golden Gate method by referring to TAL Effector Nucleotide Targeter 2.0 (https://tale-nt.cac.cornell.edu/node/add/single-tale). If the CRISPRa system is used as an activation tool, it can be designed by the same method as the editing tool described above.

These activation tools can be introduced into target cells using the same techniques as the editing tools described above.

Process of Adding a Tag to the Gene Sequence of a Target Gene

The method for detecting the expression of a target gene can be selected as appropriate depending on the characteristics of the target gene and includes, for example, a method using an antibody reactive to a membrane protein, or a method for detecting a secreted protein by its enzymatic activity.

In one embodiment, tissue stem cells with the edited gene sequence can be selected by using a tag added to the gene sequence. For example, by adding a tag sequence to the target gene that is expressed only when the target gene is edited to the desired normal gene, only cells that express the target gene normally can be accurately selected at the single cell level. Such tag sequences include, without limitation, peptides or polypeptide tags (FLAG, HA, His, Myc, V5, S, Trx, etc.), reporter genes (luciferase, etc.), fluorescent proteins (GFP, RFP, Venus, etc.), and such. The Venus sequences can be created by introducing a homologous gene of the target gene into a pVenus vector, with reference to the method described in the literature (Matsubara, Y, et al. Sci Rep, 4: 5043, 2014), and used as a template DNA. Such tags can be detected using labeled antibodies, fluorescence or luminescence, and or such. In the present invention, small molecules such as peptides (FLAG, HA, etc.) that are less damaging to cells and the HiBiT system (Promega) described below are preferred from the viewpoint of live cell detection and mutagenesis efficiency.

In one embodiment, it may be desirable for the tag to be a molecule as small as possible, such as a peptide, in view of limiting unforeseen effects in the target cell. A non-limiting example of such a tag is the HiBiT system (Promega) or such. This system uses an 11-amino acid peptide tag (HiBiT) as a tag and a luciferase fragment (LgBiT) that binds to the tag for detection, thus minimizing the effect on target cells and enabling highly sensitive detection of target cells.

The tagging tool for adding such a tag sequence to the target gene sequence (tagging tool) is not particularly limited and may, for example, be the same as the editing tool described above. In one embodiment, the tagging tool includes a guide RNA targeting the target gene, a Cas nuclease, and a template DNA for insertion of the tag sequence. Such a tagging tool can also be introduced into the target cell by the same method as the editing tool and the activation tool described above. The position at which the tag is introduced can be any site at the N terminus, C terminus, or central portion of the target gene. It is also preferable to introduce the tag at the C-terminal side from the viewpoint that the expression of the intact protein of the target gene can be detected.

ACT Genome Editing

In some embodiments, the above steps of editing the gene sequence of a target gene in a tissue stem cell, activating the gene expression of the target gene in the tissue stem cell, and adding a tag to the gene sequence of the target gene are performed in combination. As used herein, “performed in combination” means that the processes from editing to selection are performed within a short period of time in which the target tissue stem cell can be maintained in vitro.

To this end, in one embodiment, the activation tool, the editing tool (correction tool), and the tagging tool can be introduced into the target cell substantially simultaneously. In the present application, the genome editing technology combining an activation tool, an editing tool (correction tool) and a tagging tool is referred to as “ATC genome editing”. For example, in ACT genome editing, one can introduce into the target cell substantially simultaneously, a ZFN, TALEN, or CRISPR/Cas-based editing tool that targets an editing site of a target gene, a ZFN, TALEN, or CRISPR/Cas-based tagging tool that targets a translation region (C-terminal side or N-terminal side or within a translation region) of the target gene, and a ZFN, TALEN, or CRISPR/Cas-based activation tool that targets a promoter site of the target gene. Here, the same system may be used for the editing tool and the tagging tool. By doing so, the introduction of multiple mutations (editing and tagging of the target gene) and the expression of the edited target gene can be performed in an extremely short time. As a result, tissue stem cells in which the target gene has been precisely edited can be accurately selected in a short period of time.

Here, “a short period of time” means any period of time during which the target tissue stem cells can be maintained ex vivo, and it is preferably one week or less, more preferably three days or less (72 hours or less), such as two days or less (48 hours or less, 36 hours or less, 24 hours or less, or 18 hours or less). Also, “substantially simultaneously” means within a few hours (for example, within 2 or 3 hours), and it is preferable to introduce the activation tool, the editing tool, and the tagging tool at the same time for simplicity of work, but the order of introduction may be changed or the time may be shifted to improve the efficiency and accuracy of gene editing and gene expression. For example, all components used for the activation tool, the editing tool, and the tagging tool may be introduced into the cell in a single electroporation, or the components used for the editing tool and the tagging tool may be introduced, and then the components used for the activation tool may be introduced after a time interval.

In the conventional genome editing technology, even in the case of gene editing using the CRISPR/Cas system or such, it is necessary to perform single cell cloning after gene editing, and to select cells in which the target gene has been edited by genotyping each clone by PCR or such. It took several days to several weeks to obtain the target cell. In addition, if a drug resistance gene or such is inserted into the target gene, the target cell can be easily obtained by selecting drug-resistant strains, but when the insertion of foreign DNA such as a drug resistance gene is performed by homologous recombination between genomic DNA and a donor vector, there have been problems such as extremely low efficiency in general. However, according to the method of the present embodiment, editing, tagging, and activation are performed substantially simultaneously, thereby enabling highly efficient selection of only cells in which the target gene has been edited in extremely short time within the time period in which tissue stem cells can be maintained in vitro.

In one embodiment, the method of the present invention makes it possible to select tissue stem cells with the edited gene sequence within 2 days, preferably within 24 hours of isolating the cells from the patient.

Methods for selecting gene-edited tissue stem cells at the single cell level include, but are not limited to, the limiting dilution method and the cell sorter method. The tissue stem cells with the edited gene sequence may be selected using a tag added to the gene sequence. In some embodiments, the tag may comprise part or all of a system that emits fluorescence or phosphorescence. The tags may utilize, for example, the HiBiT system from Promega (Wisconsin, USA), as described above.

Tissue Stem Cells

In the methods of the present invention, editing of the gene sequence is usually performed ex vivo in isolated tissue stem cells.

Tissue stem cells (also referred to as tissue-specific stem cells or somatic stem cells) can be classified based on the site of origin of the cells, such as the skin system (epidermal stem cells, hair follicle stem cells, etc.), digestive system (pancreatic (common) stem cells, liver stem cells, etc.), bone marrow system (hematopoietic stem cells, mesenchymal stem cells (including muscle satellite cells or such), etc.), and the nervous system (neural stem cells, retinal stem cells, etc.). Although the tissue stem cells to which the method of the present invention can be applied are not particularly limited, the method is in particularly effective for the bone marrow system, specifically in hematopoietic stem cells, for reasons such as the pathology of the disease, established transplantation methods, and tissue fixation of the transplanted cells.

Tissue stem cells may be obtained by isolating from cells of each derived tissue or by inducing differentiation from more undifferentiated pluripotent stem cells, such as ES cells and iPS cells, but in the method of the present invention, it is preferable to isolate from cells of each derived tissue from the safety point of view and the like. For example, hematopoietic stem cells can be obtained by isolation from bone marrow, umbilical cord blood, or peripheral blood. Such isolation methods are not particularly limited and methods known to those skilled in the art can be used. For the method for obtaining hematopoietic stem cells, for example, the methods described in the literature (Bak R O, et al. Nat Protoc. 2018; 13(2):358-376, Forraz N, et al. Stem Cells. 2004; 22(1):100-108.) can be referred to. Specifically, for example, the bone marrow fluid is centrifuged (1500 rpm, 5 minutes, 4° C.) to remove the supernatant, and then 1 ml of erythrocyte lysis medium (Takara Bio, 786-649) and 9 ml of RPMI-1640 medium (Fujifilm Wako Pure Chemical, 189-02025) are added, followed by further centrifugation (1500 rpm, 5 minutes, 4° C.) to remove red blood cells, and cells washed with the HBSS+ medium are subjected to selection using an antibody by lineage negative selection. For lineage negative selection, the MagniSelect (trademark) Human Hematopoietic Lineage Depletion Kit (Thermofisher Scientific, 8804-6836-74) can be used.

Extraction of mouse hematopoietic stem cells can be performed with reference to the methods described, for example, in the literature (Ema H, et al. Nat Protoc. 2006; 1(6):979-87, Gundry M C, et al. Cell Rep. 2016; 17:1453-61, Hetzel M, et al. Blood. 2018; 131(5):533-545). Specifically, bilateral femurs and tibias of mice are harvested and collected into a 6 cm dish containing about 6 ml of the HBSS+ medium with 2% fetal bovine serum (Fujifilm Wako Pure Chemicals, 082-09365). The bilateral bone ends are separated, and a 25G needle is inserted into the excised fragments, followed by extrusion with the HBSS+ medium, and the cells are collected through a 70 μm nylon cell strainer (FALCON, 352350). The supernatant is removed by centrifugation (1500 rpm, 5 minutes, 4° C.), and 1 ml of erythrocyte lysis medium (Takara Bio, 786-649), RPMI-1640 medium (Fujifilm Wako Junyaku, 189-02025) and 9 ml of 10% fetal bovine serum are added, and red blood cells are removed by performing further centrifugation (1500 rpm, 5 minutes, 4° C.). Cells washed with the HBSS+ medium are subjected to lineage negative selection using antibodies. The following antibodies are administered at a dose of 2 μl per 1.0×10⁷ cells (Biotin anti-mouse Ter-119/Erythroid Antibody (BioLegend, 79748), Biotin anti-mouse CD11b Antibody (BioLegend, 79749), Biotin anti-mouse Ly-6G/Ly-6C (Gr-1) Antibody (BioLegend, 79750), Biotin anti-mouse NK-1.1 Antibody (BioLegend, 108703), and Biotin anti-mouse CD45R/B220 Antibody (BioLegend, 79752), Biotin anti-mouse CD127 (IL-7Ra) Antibody (BioLegend, 135005), Biotin anti-mouse CD3 Antibody (BioLegend, 79751)). The cells are reacted with the antibodies on ice for 60 minutes, and after washing, suspended in 500 μl of 0.5% fetal bovine serum+1% PBS solution per 1.0×10⁷ cells, followed by addition of 50 μl of Dynabeads (trademark) M-280 Streptavidin (Invitrogen, 11205D). The cells are further reacted on ice for 60 minutes, and negative cells that have not bound to the antibodies are collected using a magnetic stand. The cells are washed with the HBSS+ medium, suspended in 100 μl of culture medium per 1×10⁶ cells, and incubated (1 to 24 hours) at 37° C. under 5% CO₂. For the culture medium, the StemSpan (trademark) SFEM medium (STEM CELL Technologies, 09600) is used, and L-glutamine (Fujifilm Wako Pure Chemicals, 073-05391), mouse stem cell factor (Fujifilm Wako Pure Chemicals, 196-15581), thrombopoietin (R&D 488-TO-005), insulin-like growth factor-2 (Cosmo Bio, 100-14), and fibroblast growth factor (Fujifilm Wako Pure Chemicals, 062-06041) are added to final concentrations of 200 mmol/L, 100 ng/ml, 50 μg/ml, 100 ng/ml, 100 ng/ml, and 1 mM, respectively.

Extraction of human hematopoietic stem cells can be performed, for example, with reference to the methods described in the literature (Bak R O, et al. Nat Protoc. 2018; 13(2):358-376; Forraz N, et al. Stem Cells. 2004; 22(1):100-108). Specifically, for example, human bone marrow fluid is centrifuged (1500 rpm, 5 minutes, 4° C.) to remove the supernatant, and 1 ml of erythrocyte lysis medium (Takara Bio, 786-649), RPMI-1640 medium (Fujifilm Wako Junyaku, 189-02025), and 9 ml of 10% fetal bovine serum are added, followed by further centrifugation (1500 rpm, 5 minutes, 4° C.) to remove the red blood cells. Cells washed with the HBSS+ medium are subjected to lineage negative selection using the antibodies. Lineage negative selection is performed using the MagniSelect (trademark) Human Hematopoietic Lineage Depletion Kit (Thermofisher Scientific, 8804-6836-74). The cells are washed with the HBSS+ medium, suspended in 100 μl of culture medium per 1×10⁶ cells, and incubated (1 to 24 hours) at 37° C. under 5% CO₂. For the culture medium, the StemSpan (trademark) SFEM II medium (StemCell Technologies, 9655) is used, with IL-6 (PeproTech, 200-06), StemRegeninl (CellagenTech, C7710), UM171 (StemCell Technologies, 72914), Flt3L (PeproTech, 300-19), thrombopoietin (PeproTech, 300-18), and human stem cell factor (PeproTech, 300-07) adjusted to achieve final concentrations of 100 ng/ml, 0.75 μM, 35 nM, 100 ng/ml, 100 ng/ml, and 100 ng/ml, respectively.

In particular, it is preferable that the tissue stem cells are tissue stem cells isolated from a patient. When the tissue stem cells whose gene has been edited by the method of the present invention are used to treat a patient, the use of tissue stem cells derived from the patient circumvents the problems of donor availability and GVHD due to transplantation. It also has the advantage of being less burdensome to the patient as the pre-treatment is mild.

The method of the present invention may further comprise the step of proliferating the selected tissue stem cells as needed.

For culture and proliferation of tissue stem cells, components and conditions known in the art for maintenance of an undifferentiated state and proliferation of cells can be used. For example, the basic medium (including inorganic salts, carbohydrates, hormones, essential amino acids, non-essential amino acids, vitamins, fatty acids, etc.) includes the SFEM medium, SFEM II, D-MEM, MEM, RPMI 1640, BME, D-MEM/F-12, Glasgow MEM, Hanks' solution, mTeSR1 and such. If necessary, the medium may contain stem cell factor, basic fibroblast growth factor (bFGF), leukocyte migration inhibitory factor (LIF), interleukin, insulin-like growth factor, transferrin, heparin, heparan sulfate, collagen, fibronectin, progesterone, selenite, B27 supplement, N2 supplement, ITS supplements, antibiotics, or such. Serum or plasma may also be added to the culture medium. Each component should be of a grade that is compatible for implantation into the patient.

In one embodiment, tissue stem cells with the edited gene sequence that have been selected and expanded as needed by the method of the present invention can be transplanted into a patient. In particular, the present invention makes it possible to genetically edit tissue stem cells isolated from a patient and transplant them into the patient in an extremely short period of time, and is therefore applicable to a variety of tissue stem cells, particularly cells that are difficult to maintain in an undifferentiated state in vitro, such as hepatocytes and endogenous tissue stem cells (satellite cells) of skeletal muscle. There are also advantages of the possibility of rapid treatment in response to changes in the patient's condition, such as the need to increase the expression level of the target gene due to stress.

Therapeutic Agents

One embodiment of the present invention also relates to a therapeutic or prophylactic agent comprising a tissue stem cell in which the genetic sequence of a target gene has been edited for use in the treatment of a disease.

The therapeutic and prophylactic agents of the present invention comprise an effective amount of tissue stem cells with the edited gene sequence for treating a disease. The tissue stem cells with the edited gene sequence can be, in particular, those within 72 hours, 48 hours, 36 hours, 24 hours, or 18 hours of isolation from an organism. The amount of tissue stem cells to achieve a therapeutic effect may be determined by a person skilled in the art, taking into account the route of administration, mode of administration, target disease, therapeutic target, target site of treatment, and the like, in order to achieve an appropriate therapeutic response.

A non-limiting example of a dosing regimen is a daily dose of 1×10⁵ to 2×10⁶ cells per kg of patient weight. The administration may be single or multiple doses, or continuous infusion. The administration may begin at a low dose and be gradually increased up to the target dose based on therapeutic efficacy. Of course, the dose may be changed to an amount outside the above range depending on the condition and urgency of the subject of treatment. The route of administration may be local or systemic, for example, parenteral delivery such as intravenous, intraportal, intramuscular, intraperitoneal, intratarget tissue, subcutaneous, or intradermal administration. Mode of administration includes parenteral administration forms such as injections, suspensions, infusions, medical hydrogels, and the like.

In addition to the tissue stem cells with the edited gene sequence, the therapeutic agent of the present embodiment may contain any additional components. Non-limiting examples of the additional components include pharmaceutically acceptable diluents, carriers, and other additives, such as saline, isotonic solutions, buffers, soothing agents, stabilizers, preservatives, antioxidants, and the like.

The therapeutic agent of the present embodiment may be used alone or in combination with other agents that are effective in treating the target disease.

In this embodiment, the tissue stem cell with the edited gene sequence can be a hematopoietic stem cell, a mesenchymal stem cell, or a liver stem cell.

From another point of view, one embodiment of the present invention relates to a method of treating or preventing a disease, comprising administering to a patient in need of treatment a tissue stem cell in which the gene sequence of a target gene has been edited. Further, one embodiment of the present invention relates to the use of tissue stem cells with the edited gene sequence in the manufacture of a medicament for use in a method of treating or preventing a disease. The characteristics of these tissue stem cells have been described above.

Potential Target Disease Groups

The genome editing technology using the methods of the present invention can be applied to a wide range of diseases, including but not limited to genetic diseases, as long as the diseases involve the regulation of target gene expression in tissue stem cell-derived cells. The methods of the present invention are effective for all forms of inheritance, whether autosomal dominant inheritance, autosomal recessive inheritance, X-linked recessive inheritance, or X-linked dominant inheritance, and are particularly suitable for application to autosomal dominant genetic diseases for which conventional gene transfer has little therapeutic effect.

In conventional gene therapy in which the target gene is supplemented or added, there are issues such as the fact that the abnormal gene remains, the incorporation site of the normal gene is uncontrollable, the expression of the introduced gene is difficult to regulate, and the diseases to which it can be applied are also limited. However, the methods of the present invention enable loss of the function of an abnormal gene or specific gene, repair of the mutation of an abnormal gene, introduction of a gene into a safe site that does not cause cancer, and regulation of the expression of a gene. Furthermore, since the cells in which the target gene is normally edited can be stably maintained in the patient's body, long-term maintenance of the therapeutic effects is expected.

Among other things, the methods of the present invention may be particularly useful for single gene diseases, in particular, diseases caused by deletion mutations/frameshifts or comparable genetic mutations.

For the diseases that may be targeted by the methods or therapeutic agents of the present invention, examples include:
immunodeficiency syndromes: for example, ADA deficiency, X-linked severe combined immunodeficiency (SCID), other SCID, Wiskott-Aldrich syndrome, chronic granulomatosis, leukocyte adhesion deficiency, familial hemophagocytic syndrome, X-linked hyper IgM syndrome, X-linked lymphoproliferative disease, X-linked agammaglobulinemia, hyper IgE syndrome, and such;
abnormal Hb diseases: for example, sickle cell disease, ß-thalassemia, and such;
metabolic diseases: for example, Gaucher disease, mucopolysaccharidosis, X-linked adrenoleukodystrophy, heterochromic leukodystrophy, marble bone disease, and such;
others: Fanconi anemia, Schwachman-Diamond syndrome, Kostmann syndrome, and such (see, for example, Richard A. Morgan et al., 2017, Cell Stem Cell, 21, 574-590).

Examples of diseases that may be targets of the methods or therapeutic agents of the present invention and their target genes are listed in Table 1 below.

TABLE 1

| | | Major | Mode of |
|---|---|---|---|
| Classification | Name of disease | causative gene | inheritance |
| Immunodeficiency | ADA deficiency | ADA | autosomal |
| syndromes | | | recessive |
| | X-linked severe | IL2RG | X-linked |
| | combined | | |
| | immunodeficiency | | |
| | (SCID) | | |
| | other SCID | | autosomal |
| | | | recessive/ |
| | | | dominant/ |
| | | | X-linked |
| | Wiskott-Aldrich | WAS | X-linked |
| | syndrome | | |
| | chronic | CYBB | X-linked |
| | granulomatosis | | |
| | leukocyte adhesion | INTGB2, | autosomal |
| | deficiency | etc. | recessive |
| | familial | UNC13D, | autosomal |
| | hemophagocytic | etc. | recessive/ |
| | syndrome | | dominant/ |
| | | | X-linked |
| | X-linked hyper IgM | CD40L | X-linked |
| | syndrome | | |
| | X-linked | SAP/ | X-linked |
| | lymphoproliferative | SH2D1A | |
| | disease | | |
| | X-linked | BTK | X-linked |
| | agammaglobulinemia | | |
| | hyper IgE syndrome | STAT3, etc. | autosomal |
| | | | recessive/ |
| | | | dominant |
| | | | (STAT3) |
| | others | | |
| Abnormal Hb | sickle cell disease | hemoglobin | autosomal |
| diseases | | | recessive |
| | β-thalassemia | hemoglobin | autosomal |
| | | | dominant |
| Metabolic | Gaucher disease | β-glucosidase | autosomal |
| diseases | | | recessive |
| | mucopolysaccharidosis | ID2S, etc. | autosomal |
| | | | recessive/ |
| | | | X-linked |
| | X-linked | ABCD1 | X-linked |
| | adrenoleukodystrophy | | |
| | heterochromic | ARSA | autosomal |
| | leukodystrophy | | recessive |
| | marble bone disease | TCIRG1, | autosomal |
| | | etc. | recessive/ |
| | | | dominant |
| Others | Fanconi anemia | FANCA, etc. | autosomal |
| | | | recessive/ |
| | | | dominant |
| | Schwachman- | SBDS | autosomal |
| | Diamond syndrome | | recessive |
| | Kostmann syndrome | HAX1 | autosomal |
| | | | recessive |

Among them, since the methods according to the present invention can obtain gene-edited tissue stem cells in an extremely short period of time, they are expected to increase the tissue establishment rate of transgenic cells more efficiently and safely by reducing, respectively, the possibility of a non-physiological cell population enriched with cells that have high proliferative capacity due to long-term culture, the possibility of decreased establishment due to advanced differentiation, and the possibility of bacterial, viral, and mycoplasma proliferation by culture. For these reasons, it is preferable to apply this technology to hematological and immunological diseases for which no treatment has been established to date, such as chronic granulomatous disease, Wiskott-Aldrich syndrome, which are thought to benefit from long-term engraftment, and diseases that develop in a dominant-negative manner, such as hyper IgE syndrome.

### EXAMPLES

The present invention will be described specifically based on the examples below, but the present invention is not limited in any way to these examples.

### Example 1: Introduction of a Gene Mutation into Mouse Hematopoietic Stem Cells

Introduction of a mutation into a target gene site was performed in mouse hematopoietic stem cells extracted from wild-type mouse femurs using the CRISPR/Cas9 system. Collection of mouse hematopoietic stem cells was performed with reference to the methods described (Ema H, et al. Nat Protoc. 2006; 1(02979-87, Gundry M C, et al. Cell Rep. 2016; 17:1453-61, Hetzel M, et al. Blood. 2018; 131(5):533-545). Specifically, bilateral femurs and tibias of mice were harvested and collected into a 6 cm dish containing about 6 ml of HBSS+ medium with 2% fetal bovine serum (Fujifilm Wako Pure Chemical, 082-09365). The bilateral bone ends were separated, and a 25G needle was inserted into the excised fragment, and the cells were extruded with the HBSS+ medium and collected through a 70 μm nylon cell strainer (FALCON, 352350). The supernatant was removed by centrifugation (1500 rpm, 5 minutes, 4° C.), and 1 ml of erythrocyte lysis medium (Takara Bio, 786-649), RPMI-1640 medium (Fujifilm Wako Junyaku, 189-02025), and 9 ml of 10% fetal bovine serum were added, followed by further centrifugation (1500 rpm, 5 minutes, 4° C.) to remove the red blood cells. The cells were washed with the HBSS+ medium and subjected to lineage negative selection using antibodies. The following antibodies were administered at a dose of 2 μl per 1.0×10⁷ cells: Biotin anti-mouse Ter 119/Erythroid Antibody (BioLegend, 79748), Biotin anti-mouse CD11b Antibody (BioLegend, 79749), Biotin anti-mouse Ly-6G/Ly-6C(Gr-1) Antibody (BioLegend, 79750), Biotin anti-mouse NK-1.1 Antibody (BioLegend, 108703), Biotin anti-mouse CD45R/B220 Antibody (BioLegend, 79752), Biotin anti-mouse CD127(IL-7Ra) Antibody (BioLegend, 135005), and Biotin anti-mouse CD3 Antibody (BioLegend, 79751). The cells were reacted with the antibodies for 60 minutes on ice, washed, and suspended in 500 μl of 0.5% fetal bovine serum+1% PBS solution per 1.0×10⁷ cells, and 50 μl of Dynabeads (trademark) M-280 Streptavidin (Invitrogen, 11205D) was added. The cells were further reacted on ice for 60 minutes, and negative cells that have not bound to antibodies were collected using a magnetic stand.

The target gene was IL2RG, and the sgRNA mixture and Cas9 recombinant protein were introduced by electroporation. Interleukin 2 receptor y chain (I12rg) is the causative gene of X-linked severe combined immunodeficiency (X-SCID). sgRNA was prepared using Alt-RTM (IDT). Alt-RTM crRNA (100 μM, IDT, custom synthesis (https://sg.idtdna.com/jp/site/)) containing the target sequence (aggattgatgttcaggcttc; SEQ ID NO.:1), tracrRNA (100 μM, IDT, 1072532), and Nuclease-free Duplex Buffer (IDT, 11-01-03-01) were mixed at 3.58 μl, 3.58 μl, and 7.84 μl, respectively, placed at 95° C. for 5 minutes, and cooled at room temperature. For the Cas9 recombinant protein, SpCas9 (IDT, 1074182) was diluted to 10 mg/ml and used. As for the details of cell culture and RNA and protein transfection methods, the nuclease recombinant protein (3.69 μl at 10 mg/ml) was added to the RNA mixture (100 μM) of sgRNA and kept at room temperature for 20 minutes. To this, 7.7 μl of 1.0×10⁶ mouse hematopoietic stem cells and a template DNA (1 mg/ml) were mixed and diluted with the OptiMEM medium (ThermoFisher Scientific, 31985070) to make 100 μl. Electroporation was performed on this mixture of cells, RNA, protein and oligo DNA using a Nepa21 pulse generator (Nepa Gene). Two rectangular electric pulses (225 V, 2 ms width, 50 ms interval) followed by five pulses (30 V, 50 ms width, 50 ms interval) were applied. To the electroporated mixture, 2 ml of the HBSS+ medium was added, followed by centrifugation at 800 rpm for 3 minutes and washing.

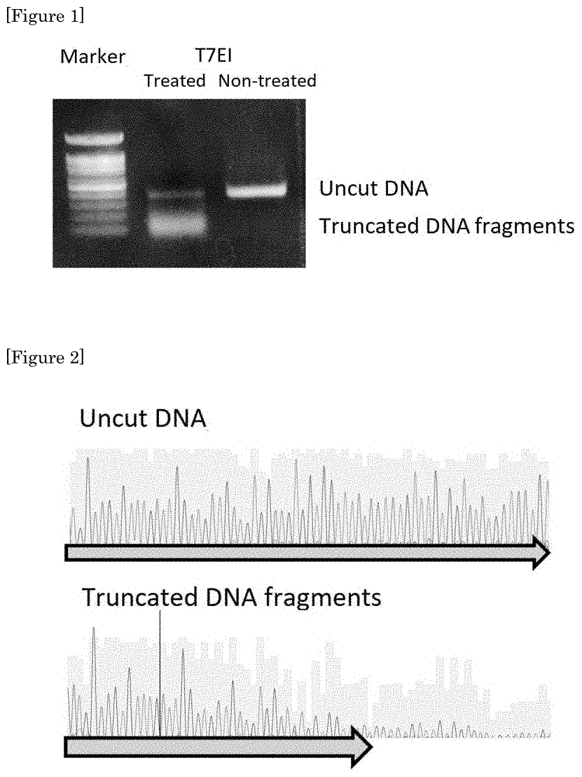

Twenty-four hours after introduction of the mutation, genomic DNA was extracted (DNeasy Blood & Tissue Kit, Qiagen, 69504), and PCR amplification (Quick Taq® HS DyeMix, Toyobo, DTM-101) was performed on a genomic DNA sequence of approximately 500 bp in length flanking the target site. The forward primer (gagctatctgtctttaggcctggag; SEQ ID NO.: 2) and reverse primer (caacctggcctacatagtgagctc; SEQ ID NO.: 3) were used. The PCR conditions were 94° C. for 2 minutes, followed by 30 cycles of 94° C. for 30 seconds/60° C. for 30 seconds/68° C. for 30 seconds. The PCR products were purified and denatured in NEBuffer2 (New England BioLab, B7002) at 95° C. for 5 minutes. This was followed by slow annealing at room temperature to form a DNA mismatch at the site of introduction of the mutation. This was followed by DNA mismatch cleavage by treatment with T7 endonuclease I (T7EI) (New England BioLab, M03025), which recognizes and cleaves only mismatch-containing DNA, at 37° C. for 15 minutes. FIG. 1 shows the electrophoresis of the PCR products treated with T7 endonuclease I on a 1% agarose gel, indicating that the formation of the DNA mismatch, i.e., introduction of the mutation, was obtained at the target site. FIG. 2 shows the sequencing results of the truncated PCR products (FASMAC, Big Dye terminator v3.1, 3130xl Genetic Analyzer). The primer used for sequencing had the nucleotide sequence of gagctatctgtctttaggcctggag (SEQ ID NO.: 4). The PCR products after the cleavage (FIG. 2, bottom) differ from the PCR products before the cleavage (FIG. 2, top) in that the sequence after the target site is illegible, indicating that the PCR products were cleaved at the same site.

### Example 2: Introduction of a Tag Sequence into a Target Gene

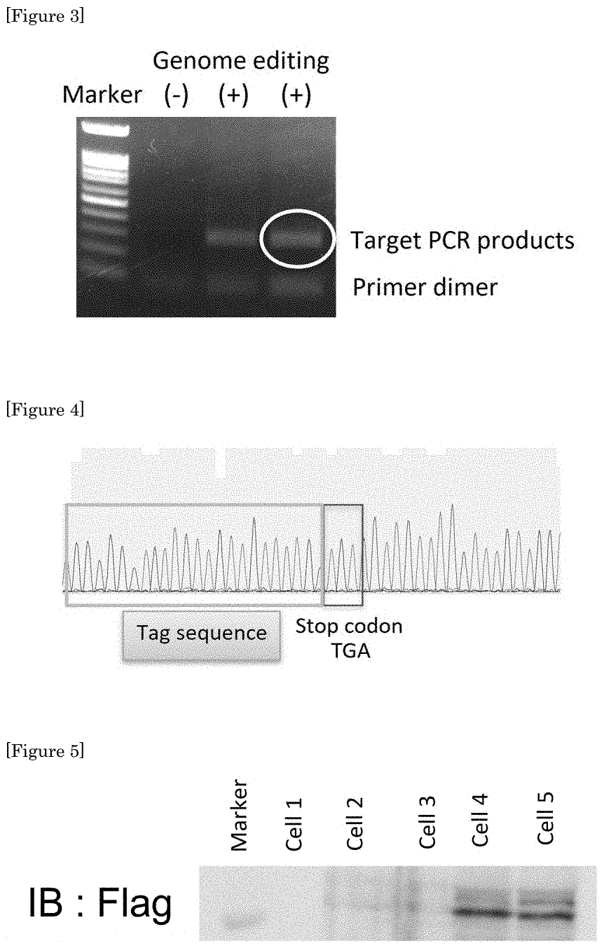

The tag sequence mutation was introduced into the target gene site using the CRISPR/Cas9 system in mouse hematopoietic stem cells extracted from wild-type mouse femurs. The target gene was IL2RG, and a mixture of sgRNA containing the target sequence (aggattgatgttcaggcttc; SEQ ID NO.: 5), the Cas9 recombinant protein, a template DNA for introducing the tag sequence (tgcattagcccttacctgcccccatgttattctctgaagccggaagcctgagcggctgcggtgatcgaaga tagcctggaacatctccttgatggaacctcaaagtctcctatagtcctaagtgac; SEQ ID NO.: 6) were introduced by electroporation. Twenty-four hours after introduction of the mutation, genomic DNA was extracted (DNeasy Blood & Tissue Kit, Qiagen, 69504), and PCR amplification was performed (Quick Taq® HS DyeMix, Toyobo, DTM-101), by designing the forward primer (gtgagcggctggcggctgtt; SEQ ID NO.: 7) to be within the tag sequence and the reverse primer (caacctggcctacatagtgagctc; SEQ ID NO.: 8) to be about 300 bp downstream. The PCR conditions were 94° C. for 2 minutes, followed by 30 cycles of 94° C. for 30 seconds/60° C. for 30 seconds/68° C. for 30 seconds. FIG. 3 shows the electrophoresis of the PCR products on a 1% agarose gel. FIG. 4 shows the sequencing result of the PCR products. The primer used for sequencing had the nucleotide sequence of caacctggcctacatagtgagctc (SEQ ID NO.: 9). These indicate that the tag sequence was introduced into the target site.

### Example 3: ACT Genome Editing

In Activation, the TAL effector is created by introducing it into a mammalian expression vector (pcDNA-TAL-VP-64 (Addgene, 47107)) by the Golden Gate method by referring to the TAL Effector Nucleotide Targeter 2.0 (https://tale-nt.cac.cornell.edu/node/add/single-tale), with reference to the method described in the literature (Matsubara, Y, et al. Sci Rep, 4: 5043, 2014).

In Correction and Tagging, when sgRNA is created using Alt-RTM (IDT), Alt-RTM crRNA (100 μM, IDT, custom synthesis (https://sg.idtdna.com/jp/site/)) containing the target sequence, tracrRNA (100 μM, IDT, 1072532), and Nuclease-free Duplex Buffer (IDT, 11-01-03-01) are mixed at 3.58 μl, 3.58 μl, and 7.84 μl, respectively, placed at 95° C. for 5 minutes, and cooled at room temperature.

In the preparation of sgRNA using a gRNA vector (Addgene No. 41824), the target sequence is introduced by inverse PCR to prepare the gRNA template DNA. The RNA is synthesized using the mMessage mMachine (trademark) T7 UTRA Transcription Kit (Ambion, AM1345), and purified using the MEGAclear (trademark) Transcription Clean-Up Kit (Ambion, AM1908) to 100 μM. If nuclease is used as the recombinant protein, SpCas9 (IDT, 1074182) and cpf1 (IDT, 1081069) are diluted to 10 mg/ml and used.

When using a nuclease as RNA, the RNA can be synthesized by the mMessage mMachine (trademark) T7 UTRA Transcription Kit (Ambion, AM1345) using SpCas9, SpCas-9-NG, dCas9, M-NMn-VP64 (Addgene #80425, #41816, #48676) on the vector as template DNA, and purified by the ethanol precipitation methods, with reference to the method described in the literature (Nishimasu H, et al. Science. 2018; 361:1259-1262. Esvelt K M, et al. Nat Methods. 2013; 10(10:1116-1121).

Next, the above tools of RNA and recombinant protein for Activation, Correction, and Tagging are simultaneously introduced into cells by electroporation to select cells in which editing and repair of the target gene has been performed. In other words, as correction, introduction of the mutation is performed using sgRNA targeting the target mutation site of the target gene, SpCas9, and the template DNA. In addition, for tagging, sgRNA targeting the C-terminal side of the protein translation region, SpCas9 or SpCas9-NG, and the template DNA are used to introduce the labeled sequence. Only if these sequences work well, the cells in which the sgRNA targeting the promoter region of the target gene induces activation of the target gene by M-NMn-VP64 or the TAL effector, and the mutation site of the target gene is precisely edited, and that are able to produce the protein will be selected based on the signal of the labeling sequence. As the labeling sequence, the above-mentioned HiBit sequence, HA sequence, GFP sequence, Venus sequence or the like is used. The Venus sequence is prepared by introducing the homologous gene of the target gene into the pVenus vector, with reference to the method described in the literature (Matsubara, Y. et al. Sci Rep, 4:5043, 2014), and used as the template DNA.

As for the details of cell culture and RNA and protein transfection methods, the nuclease (3.69 μl for 10 mg/ml of recombinant protein and 2 μl for 250 ng/pi of RNA) is added to the RNA mixture (100 μM) of sgRNA and kept at room temperature for 20 minutes. To this, 7.7 μl of 1.0×10⁶ tissue stem cells and the template DNA (1 mg/ml) are mixed and diluted with the OptiMEM medium (ThermoFisher Scientific, 31985070) to make 100 μl. Electroporation is performed on this mixture of cells, RNA, protein and oligo DNA using the Nepa21 pulse generator (Nepa Gene). Two rectangular electric pulses (225 V, 2 ms width, 50 ms interval) followed by five pulses (30 V, 50 ms width, 50 ms interval) are applied. To the electroporated mixture, 2 ml of the HBSS+ medium is added, followed by centrifugation at 800 rpm for 3 minutes and washing. The cells are cultured for 24 hours in the same medium used for tissue stem cell collection. Further, cells in which gene editing has been precisely performed are selected.

### Example 4: Introduction of a Target Gene Mutation (Correction) and a Tag Label Insertion (Tagging) into Human Fetal Kidney Cancer Cells

Introduction of the targeted mutation into the target gene site and introduction of the HiBIT (Flag addition) sequence as a tag sequence were performed in a human fetal renal carcinoma cell line (293FTcell (Thermofisher Scientific)) using the CRISPR/Cas9 system. The target gene was RBP (TruB1), and a mixture of sgRNA containing the target sequence (CTGCGCTGTCTAGAGTCCCT, SEQ ID NO.: 10; CAAAAGTATGGCCGCTTCTG, SEQ ID NO.: 11), the Cas9 recombinant protein, a template DNA for introducing the tag sequence (GACCAAGAGGAAAAGCAGACTTTTGAAATGTGGCATGAGGACTCTGT AGTGTGAGCCGACGAGGTTCTGTGGTGATTGGAATTGGAAGCGGAAC AAAAA, SEQ ID NO.: 12; CAGCGTGCACCTCCACGATGAAACAGGTCTGGGCTACAAAAGTATGG CCGCTTCTGTGAGCGGCTGGCGGCTGTTCAAGAAGATTAGCGACTAC AAAGACCATGACGGTGATTATAAAGATCATGACATCGATTACAAGGAT GACGATGACAAGGCCGCTTCTGAGGCGGCGGTGGTGTCTTCGCCGTC TTTGAAAACAG, SEQ ID NO.: 13) were introduced by electroporation.

After introduction of the mutation, Nano-Glo luminescence was detected using the Nano-Glo® HiBiT Lytic detection System (Promega N3030), and enrichment was performed by continuing culture of only those cells that were positive. Proteins were extracted from these representative cells (RIPA buffer, Nacalaitesk 16488-34), and Western blotting was performed using a Flag antibody (MBL, FLA-1). FIG. 5 shows the results of Western blotting with the Flag antibody. It shows that the tag sequence was well inserted in some cells, functioning as a peptide tag.

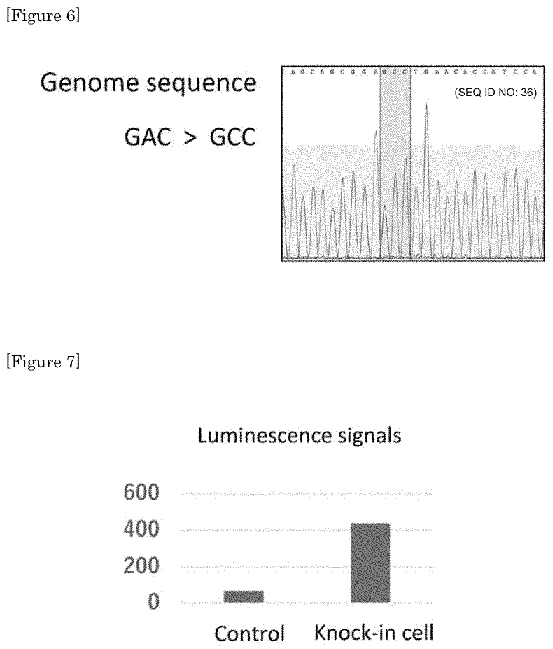

Genomic DNA was extracted from these cells by cloning (DNeasy Blood & Tissue Kit, Qiagen, 69504). PCR amplification was performed (Quick Taq® HS DyeMix, Toyobo, DTM-101), by designing the forward primer (GTTTTGAAAATGCCATCCCC, SEQ ID NO.:14) to be about 200 bp upstream of the mutation site and the reverse primer (AGAAATAGCTACTTTTATGT, SEQ ID NO.:15) to be about 300 bp downstream. The PCR conditions were 94° C. for 2 minutes, followed by 30 cycles of 94° C. for 30 seconds/60° C. for 30 seconds/68° C. for 30 seconds. FIG. 6 shows the sequencing results of one of the PCR products (FASMAC, Big Dye terminator v3.1, 3130xl Genetic Analyzer). The forward primer (SEQ ID NO.: 14) was used as the primer for sequencing. It shows that the mutation was introduced into the target sequence at the target site.

### Example 5: Introduction of a Target Gene Mutation (Correction) and Insertion of a Tag Label (Tagging) into Human Fetal Kidney Cancer Cells

The target mutation (removal of exon2) and the HiBIT sequence as a tag sequence were introduced into a target gene site and a termination codon was introduced immediately downstream to it in a human fetal renal carcinoma cell line (293FTcell (Thermofisher Scientific)) using the CRISPR/Cas9 system. The target gene was RBP (TruB1), and a mixture of sgRNA containing the complementary strand of the target sequence (TCCCCTTTTCCTCCCAAGTT, SEQ ID NO.: 16; TTTCTCTCATAGAAGCTGGA, SEQ ID NO.: 17), the Cas9 recombinant protein, and a template DNA (CTAGTAATGAGTCATAGTCCTTAACATGTAAGTTGTGTATATACTGTG TGTGTGAGCGGCCGGTGTTCAAGAGATAGATAGCTTATAGAAGCTGG AATGCCTTCTCCAG AATGGACCAAGAGGAAAAAGCAGAC, SEQ ID NO.: 18) were introduced by electroporation.

After introduction of the mutation, detection of the Nano-Glo luminescence was performed using the Nano-Glo® HiBiT Lytic detection System (Promega N3030). FIG. 7 shows the results of Lytic detection. It shows that the luciferase activity was increased in knock-in cells.

### Example 6: Introduction of a Target Gene Mutation (Correction) and Insertion of a Tag Label (Tagging) into Human Fetal Kidney Cancer Cells and Activation (Activation)

The target mutation was introduced into the target gene site and the HiBIT sequence as a tag sequence was introduced into a human fetal renal carcinoma cell line (293FTcell (Thermofisher Scientific)) using the CRISPR/Cas9 system, and activation was then performed. The target gene was STAT3, and a mutant STAT3 cell line was first established. In this step, a mixture of sgRNA containing the complementary strand of the target sequence (GTTGTGGTGATCTCCAACAT, SEQ ID NO.: 19), the Cas9 recombinant protein, a template DNA for introducing the mutation (CCCAGCTCCAGCCCCACTCCTTGCCAGTTGTGTAAGATCTCCACATC TGTCCAGATGCCAAATGCCTGGGCGTCCATCCTGTGGTA, SEQ ID NO.: 20) were introduced by electroporation.

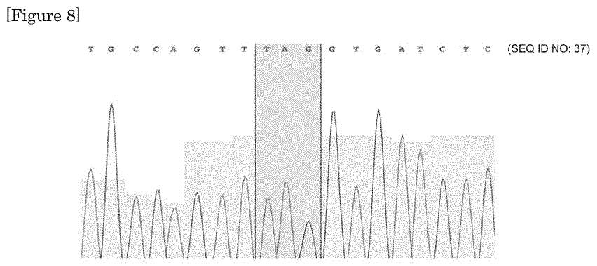

After introduction of the mutation, the cells with the introduced mutation were cloned by a limiting dilution method. The genomic DNA of the cloned cells with the introduced mutation was extracted and sequenced to confirm the mutation. FIG. 8 shows the results of this sequencing. In this PCR, the forward primer (GCAGCAGGTGTGGTTTATGG, SEQ ID NO.: 21) and reverse primer (ACCATCCCTCATCTAAACAA, SEQ ID NO.: 22) were used, and the PCR conditions were 94° C. for 2 minutes, followed by 30 cycles of 94° C. for 30 seconds/60° C. for 30 seconds/68° C. for 30 seconds. Sequencing was performed using the forward primer.

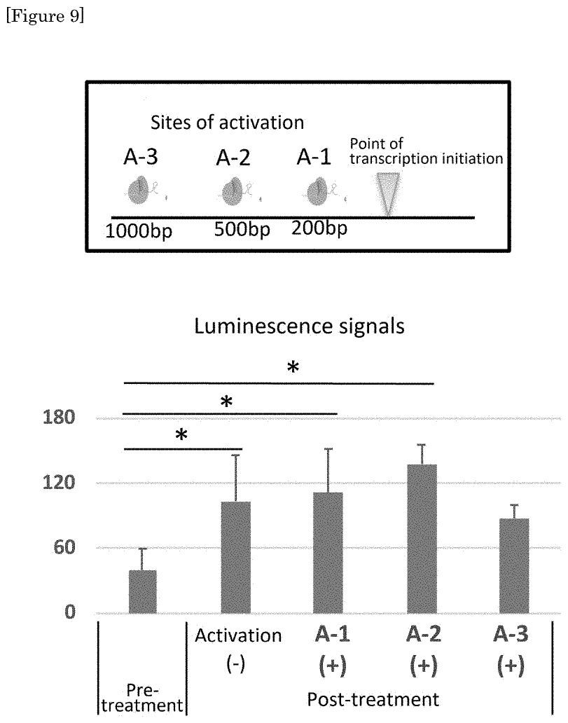

For the mutant cells obtained here, the sgRNA to undo the mutation (same as SEQ ID NO.: 19) and sgRNA for addition of the tag sequence (comprising TGCGCTACCTCCCCCATGTG as the target sequence; SEQ ID NO.:23), the Cas9 recombinant protein, a template DNA to undo the mutation (CCCAGCTCTCAAGTCCACTCTCCGCGCAGACACTCTCTGCAGTTGTG TGGTGATCTCCAAATGCCTGGGCGTCCATCCTGTG; SEQ ID NO.: 24) and a template DNA for introducing the tag sequence (CCCTCACCTTGTGACATGGAGTTGACTCTCGAGTCGTCGTCTCGATG TGTGAGCGGCTCGTCGTCGTCTTCAAGAGATAGCTGAGAGCTCTGGA GACGAGCTGACTCGAAAGATACGACTGAGGCGCCTACC; SEQ ID NO.:25) were introduced by electroporation. After the introduction of the mutation, the STAT3 gene was activated. The sgRNAs targeting sites at 1000 bp, 500 bp, and 200 bp upstream of the transcription initiation factor of the STAT3 gene, respectively (UUUUGUAGUAGUAGGAGAAUCUC, SEQ ID NO.: 26; UUUAAAAAAUGAGUGUGGCA, SEQ ID NO.: 27; and UCAAGGCCACCCUGGGCAAC, SEQ ID NO.: 28, respectively) and the mRNA obtained from the dCas9-VP64 vector (addgene, #47107) were transfected using the Fugene HD Transfection reagent (Promega, E2311), and detection of the Nano-Glo luminescence was performed 24 hours later using the Nano-Glo® HiBiT Lytic detection System (Promega N3030). For the mRNA, RNA was synthesized by mMESSAGE mMACHINE T7 Ultra (Invitrogen, AM1345) using the dCas9-VP64 vector as a template, and purified using the MEGA Clear Transcription clean kit (Invitrogen, AM1908). FIG. 9 shows the results of lytic detection. The luciferase activity of HiBIT was increased by activation, indicating that the mutation has been introduced and the changes are accentuated by gene activation.

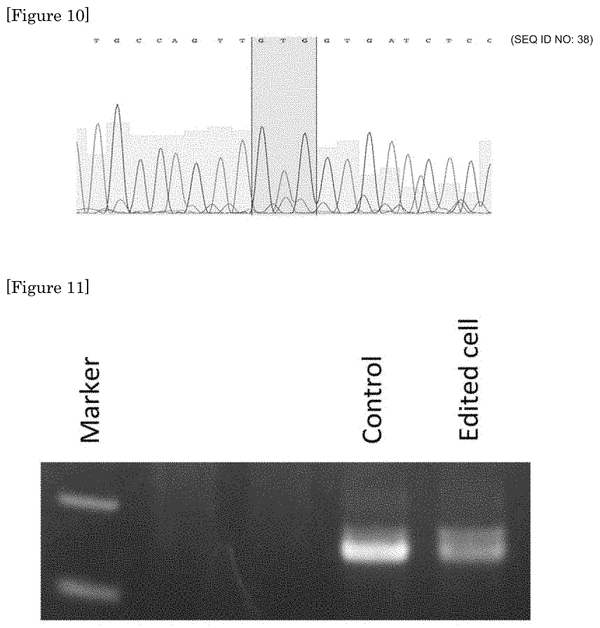

In addition, the cells were subjected to PCR using a forward primer (GCAGCAGGTGTGGTTTATGG; SEQ ID NO.: 21) and a reverse primer (ACCATCCCTCATCTAAACAA; SEQ ID NO.: 22), and the efficiency of reverting the mutation was examined by sequencing the products using the forward primer. FIG. 10 shows the sequencing results in this case. There is a waveform overlap in the target sequence, indicating that the cells with the mutation reverted are included.

### Example 7: Gene Mutation Repair and Introduction of a Tag Sequence in Mouse Hematopoietic Stem Cells

SCID mouse hematopoietic stem cells were extracted from the femur of C.B-17/Icr-SCID mice (Clare Japan) carrying the Prkdc mutation, and the CRISPR/Cas9 system was used to repair the target gene mutation and to introduce the tag sequence into the target gene. The target gene was Prkdc, and the sgRNA for repairing the mutation (comprising gcuuagcguauuuuauguug; SEQ ID NO.: 29), sgRNA for adding the tag sequence (comprising acaccacagacuuuacaucc; SEQ ID NO.: 30), the Cas9 recombinant protein, a template DNA for repairing the mutation (gatcatggatctcaagaataatgtaacggaaagatggtgatctcacacaatacaatacgcatgcatgca tgctaagagttagcaggggccaacccagctgt; SEQ ID NO.: 31) and a template DNA for introducing the tag sequence (cagaccccaatatccttggcaggacttgggaaggatgggagccctggatgCGGGACCACATGG TGCTGCACGAGTACGTGAACGCCGCCGGCATCACATAAtaaagtctgtggtg tcaccaatc ataaagcattctgtctccgagaggacc; SEQ ID NO.: 32) were introduced by electroporation. GFP-11 was used as the tag sequence.

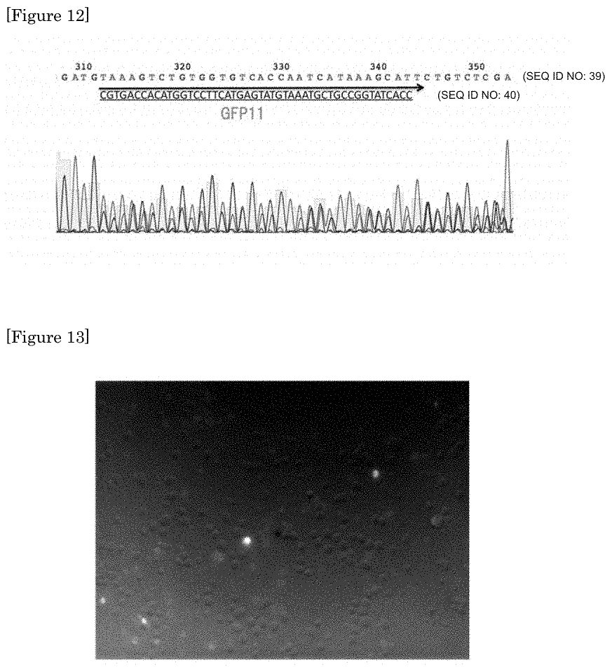

Genomic DNA was extracted 48 hours after introduction of the mutation (DNeasy Blood & Tissue Kit, Qiagen, 69504), and PCR amplification was performed (Quick Taq® HS DyeMix, Toyobo, DTM-101), using a forward primer (CAACTTGAATTCACAGTCATGAGTGAC; SEQ ID NO.: 33), and a reverse primer (GAGGTCCTCTCGGAGACAGAATG; SEQ ID NO.: 34). The PCR conditions were 94° C. for 2 minutes, followed by 30 cycles of 94° C. for 30 seconds/60° C. for 30 seconds/68° C. for 30 seconds. FIG. 11 shows the electrophoresis of the PCR products on a 1% agarose gel. FIG. 12 shows the sequencing result of the PCR products. The forward primer (SEQ ID NO.: 33) were used as the primer for sequencing. These showed overlap of the target tag sequence at the target site, indicating that the tag sequence was introduced with about 50% efficiency.

Furthermore, GFP1-10 was introduced into the cells 48 hours after introduction of the mutation using adeno-associated virus, and the cells were observed 24 hours later. The adeno-associated virus was created and transduced using the AAVpro® Helper Free System (Takara, 6673) to transduce GFP1-10 (SEQ ID NO.: 35) into pAAV-CMV (Takara, 6673). FIG. 13 shows the fluorescence of these cells. It is shown that GFP-11 is introduced into the cells and that there are cells that have obtained GFP fluorescence as a result of the binding to GFP1-10 derived from AAV.

The present specification shows the preferred embodiments of the present invention, and it is clear to those skilled in the art that such embodiments are provided simply for the purpose of exemplification. A skilled artisan may be able to make various transformations, and add modifications and substitutions without deviating from the present invention. It should be understood that the various alternative embodiments of invention described in the present specification may be used when practicing the present invention. Further, the contents described in all publications referred to in the present specification, including patents and patent application documents, should be construed as being incorporated by reference in the same manner as the contents clearly written in the present specification.

### INDUSTRIAL APPLICABILITY

The present invention enables early radical therapeutic methods for blood, immune diseases, and such, for which conventional treatment means has been insufficient.

1. A method for producing a gene-edited tissue stem cell, comprising editing the gene sequence of a target gene in the tissue stem cell and selecting the tissue stem cell with the edited gene sequence.

2. The method according to claim 1, further comprising adding a tag to the gene sequence of the target gene and/or activating gene expression of the target gene in the tissue stem cell.

3. The method according to claim 1, wherein the tissue stem cell is a tissue stem cell isolated from a patient.

4. The method according to claim 1, wherein the editing of the gene sequence is performed ex vivo.

5. The method according to claim 1, wherein the editing of the gene sequence is performed using a CRISPR/Cas system or a TALEN system.

6. The method according to claim 1, wherein the target gene is a gene that is not steadily expressed in tissue stem cells.

7. The method according to claim 2, wherein the activation of the gene expression is performed using the CRISPRa system or the TALEN effector system.

8. The method according to claim 2, wherein the tissue stem cell with the edited gene sequence is selected using the tag added to the gene sequence.

9. The method according to claim 2, wherein the step of editing the gene sequence of the target gene in the tissue stem cell and the step of adding a tag to the gene sequence of the target gene are performed simultaneously.

10. The method according to claim 1, wherein the tissue stem cell with the edited gene sequence is selected within 24 hours of isolating the cell from the patient.

11. The method according to claim 1, wherein the selected tissue stem cell maintains an undifferentiated state.

12. The method according to claim 1, further comprising proliferating the selected tissue stem cell.

13. The method according to claim 1, further comprising transplanting the selected tissue stem cell into the patient.

14. The method according to claim 1, wherein the tissue stem cell with the edited gene sequence is a cell for use in treating a disease.

15. The method according to claim 14, wherein the disease is a hematological disease or an immunological disease.

16. The method according to claim 15, wherein the hematological disease or immunological disease is selected from the group consisting of ADA deficiency, X-linked severe combined immunodeficiency (SCID), other SCID, Wiskott-Aldrich syndrome, chronic granulomatosis, leukocyte adhesion deficiency, familial hemophagocytic syndrome, X-linked hyper IgM syndrome, X-linked lymphoproliferative disease, X-linked agammaglobulinemia, hyper IgE syndrome, sickle cell disease, and β-thalassemia.

17. The method according to claim 1, wherein the target gene is selected from the group consisting of ADA, IL2RG, WAS, CYBB, INTGB2, UNC13D, CD40L, SAP/SH2D1A, BTK, STAT3, and hemoglobin.

18. A therapeutic agent for the treatment of a hematological disease or an immunological disease, comprising hematopoietic stem cells within 48 hours of isolation from an organism in which the gene sequence of the target gene has been edited.

19. The therapeutic agent according to claim 18, wherein the hematological disease or immunological disease is selected from the group consisting of ADA deficiency, X-linked severe combined immunodeficiency (SCID), other SCID, Wiskott-Aldrich syndrome, chronic granulomatosis, leukocyte adhesion deficiency, familial hemophagocytic syndrome, X-linked hyper IgM syndrome, X-linked lymphoproliferative disease, X-linked agammaglobulinemia, hyper IgE syndrome, sickle cell disease, and β-thalassemia.

20. The therapeutic agent according to claim 18, wherein the target gene is selected from the group consisting of ADA, IL2RG, WAS, CYBB, INTGB2, UNC13D, CD40L, SAP/SH2D1A, BTK, STAT3, and hemoglobin.

21. A method for producing a gene-edited tissue stem cell, comprising editing the gene sequence of the target gene in the tissue stem cell,
adding a tag to the gene sequence of the target gene,
activating gene expression of the target gene in the tissue stem cell, and
selecting the tissue stem cell with the edited gene sequence,
wherein the step of editing the gene sequence of the target gene in the tissue stem cell and the step of adding a tag to the gene sequence of the target gene are performed simultaneously using a CRISPR/Cas system or a TALEN system.

22. The method according to claim 21, wherein the target gene is a gene that is not steadily expressed in tissue stem cells, and
wherein the tag-labeled target protein of interest is produced by activating the gene expression of the target gene only in cells in which the gene sequence of the target gene has been edited and the tag has been added, and the tag is used to select the tissue stem cell with the edited gene sequence.

23. The method according to claim 21, wherein the tissue cell is a tissue stem cell isolated from a patient, and the tissue stem cell with the edited gene sequence is selected within 48 hours of isolating the cell from the patient.
